(19) United States
(12) Patent Application Publication
(10) Pub. No.: US 2022/0226580 A1
(43) Pub. Date: Jul. 21, 2022

(54) Drug Delivery Device

(71) Applicant: Sanofi, Paris (FR)

(72) Inventors: Michael Jugl, Frankfurt am Main (DE); Stefan Blancke, Frankfurt am Main (DE); Axel Teucher, Frankfurt am Main (DE); Jörg Bigalke, Frankfurt am Main (DE); Maurice Toporek, Frankfurt am Main (DE)

(21) Appl. No.: 17/617,099

(22) PCT Filed: Jun. 12, 2020
(86) PCT No.: PCT/EP2020/066373

§ 371 Date: Dec. 7, 2021

(30) Foreign Application Priority Data
Jun. 13, 2019 (EP) 19305751.0

(51) Int. Cl. A61M 5/315; A61M 5/24; A61M 5/20

(52) U.S. Cl. CPC A61M 5/3157; A61M 5/2455; A61M 5/31511; A61M 5/3158; A61M 5/20; A61M 5/31568; A61M 2205/582; A61M 2205/3569; A61M 2205/502; A61M 2205/18; A61M 2205/581; A61M 2205/583; A61M 2205/8206

(57) ABSTRACT

A drug delivery device comprising a first module comprising a zinc-air cell located at one end of the first module, a second module for attachment to the one end of the first module and comprising electronics, and activation means for activating the zinc-air cell and being designed such that at least one air hole is created in an air tight sealing of the zinc-air cell when the second module is attached to the one end of the first module.

### CROSS REFERENCE TO RELATED APPLICATIONS

The present application is the national stage entry of International Patent Application No. PCT/EP2020/066373, filed on Jun. 12, 2020, and claims priority to Application No. EP 19305751.0, filed on Jun. 13, 2019, the disclosures of which are incorporated herein by reference.

### TECHNICAL FIELD

The present disclosure relates to a drug delivery device.

### BACKGROUND

A variety of diseases exists that require regular treatment by delivery, particularly injection of a medicament. Such injection can be performed by using injection devices, which are applied either by medical personnel or by patients themselves.

### SUMMARY

Drug injection devices particularly for usage by patients themselves may be equipped with electronics for user assistance. WO2016/193122A1 discloses for example a pen-formed drug delivery device comprising a drug reservoir with an outlet, a detachable cap adapted to cover the drug reservoir outlet portion in a mounted position on the device body, and drug expelling means comprising dose setting means allowing a user to set a dose amount of drug to be expelled, the set dose printed on a rotatable scale drum being shown in a window. The device is provided with electronic circuitry comprising sensor means adapted to capture a property value related to the dose amount of drug expelled from the reservoir by the expelling means during an expelling event, processor means adapted to determine dose amounts based on captured property values, storage means adapted to store at least one dose amount, display means adapted to display dose related information, e.g. a determined dose amount and a related time value, and a power source. The power source is in the form of a zinc-air battery, which can be deprived of air and thus be inactive until first use. The zinc-air battery comprises a number of air holes in communication with the exterior through one or more channels formed between the battery and openings formed in the pen body exterior surface. The pen cap is provided with sealing surface portions adapted to seal the openings when the cap is in a fully mounted position. When the cap is removed fully or partly from the device body the channel openings are exposed and communication is established between the battery air holes and the exterior, this allowing oxygen-rich air to enter the battery whereby the above-described chemical process starts and a voltage is generated, this allowing the electronic circuitry to power up and to register an expelled dose which then may be displayed in the display means. In one aspect the present disclosure provides a modularization of a drug delivery device with a first and/or a second module, whereby the first and/or second modules are configured for, when aggregated, building up an electronically controlled and/or electronically monitored pen-shaped injection device and to thereby provide functionality for electronically controlled and/or monitored expelling of a liquid drug formulation to an external administration site, the modularization comprising:
a first module including a liquid drug filled reservoir, a first, downstream, portion of a liquid drug expelling mechanism configured to expel a portion of liquid drug from the liquid drug reservoir when actuated and a self-contained electrical energy source as, in particular, an electrochemical cell or battery, and/or
a second module including a second, upstream, portion of the liquid drug expelling mechanism and an electronic circuitry, the electronic circuitry being configured for controlling and/or monitoring of the state and/or operation of any of the first or second portion of the liquid drug expelling mechanism;
whereby electromechanical interfaces are provided on the first and/or second modules, the electromechanical interfaces being configured to:
   releasable connect the first module and the second module to thereby form a connected mechanical structure and, in more particular, a rigidly connected mechanical structure;
   to operationally connect the expelling mechanism portion of the first module to the expelling mechanism portion in the second module; and
   to provide an electrical path for supplying energy from the electrical energy source aggregated in the first module to the electronic circuitry included in the second module.

Certain aspects of this disclosure may provide a drug delivery device having a first and a second module. Further certain aspects of this disclosure may provide a drug delivery device having a first module, which is configured so that it can be coupled to a second module for building up a pen-shaped injection device. Yet further certain aspects of this disclosure may provide a drug delivery device having a second module, which is configured so that it can be coupled to a first module for building up a pen-shaped injection device.

An “injection device” as mentioned before may be considered as a more specific drug delivery device insofar as it includes an expelling mechanism as well as a liquid drug reservoir and thereby allows the user to expel liquid drug therefrom. When fed to a cannula, an injection of the expelled liquid drug into a human or veterinarian body may be effected. The injection may be, for example, sub-cutaneous, intra-muscular or intravenous according to the common understanding of these terms in the medical field. In instances, an injection device may be a dose-controlled injection device for allowing a user to expel a predetermined volume of the liquid drug from the reservoir. In instances, a dose-controlled injection device may be a multi-shot dose-controlled injection device to thereby allow a user to perform multiple dose-controlled expelling operations from the same medicament container. Typical examples thereof can be found in the various diabetes pen injection devices.

The term “modularization” as used before and hereinafter shall be understood to apply to every single module as a standalone device as well as to the aggregation of two or more modules. When, in such “modularization”, the scope is semantically limited to a single module or an incomplete aggregation of modules, any reference to a remainder module or remainder aggregation of modules shall be interpreted in terms of interfacing requirements emerging therefrom relative to the single module or an incomplete aggregation of modules in consideration.

The qualifiers “upstream” and “downstream” used before in relation with an expelling mechanism shall be understood to attach to the concept of a drivetrain implemented therein and to the flow of mechanical force or power along that drivetrain. In particular, a “downstream” located member of the drivetrain is expected to follow the operation, movement or actuation of an “upstream” member during normal expelling operation. Apparently, “upstream” and “downstream” are relative qualifiers. In a relation between two drivetrain members, the “upstream” located one is closer to the source of mechanical power which causes the expelling drivetrain to operate for expelling.

The disclosed modularization of an electronically controlled and/or monitored injection device may be applied for improving the coherence of component lifetime to thereby reduce amount in waste and costs. In particular, the modularization may be implemented in a way that collects several components with a rather reduced lifetime or expected expiration time into one of the modules. Preferably, the drug reservoir and the electrical energy source may be allocated together to the same module whereas the electronic circuitry may be allocated entirely or to an essential extent to another module. This allocation may allow replacement the module comprising the drug container and the electrical energy source at the same time thereby maintaining essential parts of the electronic circuitry with the other module for subsequent re-use. It is an underlying consideration that multiple administration liquid drug injection devices have shelf lifetime of several weeks after first use due to potential risk of medicament degradation which approximately matches with the typical period of power delivery after activation for standard zinc-air cells. On the other hand, for an electronic circuitry a more elongated time of use is desired at least for cost reasons. On the other hand it may be found preferable to keep a short lifetime module free from electronic components to avoid application of waste restrictions beyond the ones applying to empty medicament containers and sharp items. Finally, an appropriate allocation of the components to the modules may help increasing therapy quality by collating operational prerequisites into one item. In an exemplary traditional situation of a re-usable injection pen with an integrated dose capturing and recording unit, in order to safeguard for appropriately captured drug administration, a user has to take care of the charging state of the battery in the dose capturing and to check sufficiency of the remainder in the drug container in view of the upcoming administrations. Failure on either end will prevent the user to perform a properly recorded dose administration as required by the therapy plan.

In embodiments, the allocation of the overall mechanical expelling drivetrain to the first module and the second module, respectively, may allocate not only the cartridge bung as the ultimate end portion to the first module but may also include the directly adjacent upstream one or more members, namely the piston rod foot and, in instances, the piston rod into the first module. This may be useful when high structural stability in force flow cycle reaching from the piston rod support along the piston rod, to the bung, the liquid, the vial and the vial support back to the piston to support. This may be especially useful when lower volumes of higher concentrated drug formulations are required to be dispensed at a high precision.

In more specific embodiments, the first and/or second module may be configured for aggregating into an axial layout whereby the axial layout has the liquid drug filled reservoir, particularly a drug container, the liquid drug expelling mechanism and the electrical energy source arranged along the longitudinal axis defined by the pen-shaped external appearance of the aggregation of the first and second module.

In more specific embodiments, the first module may include a standard medicament cartridge with a liquid drug formulation included in a section of a glass vial, the section of the glass vial being separated from the exterior space by a rubber bung seal, the rubber bung seal being movable along a cylindrical section of the glass vial as the ultimate end of the liquid drug expelling mechanism portion in the first module for transforming mechanical drive force in the expelling mechanism into liquid pressure in the separated glass vial section.

In more specific embodiments, the electrical power source may be a button cell located adjacent to the rubber bung seal surface that faces to the exterior. In more particular embodiments, the button cell may be configured to comprise a ridged external structure suitable for transferring an axial load from an abutting drive train member into the rubber bung seal. In an even more particular embodiment, a flat-cylindrical button cell may be arranged to contact the externally facing end of the rubber bung seal with one flat circular pole and to provide the opposite circular pole face as an incoming load plate to an adjacent upstream drivetrain element of the overall expelling drivetrain. In an alternative thereto, the external edge surrounding the opposite pole may be used as the input load support for an upstream drivetrain element in order to avoid promotion of mechanical load over the seal of the button cell or through its internal structure.

In embodiments, the upstream drivetrain element abutting against the button cell may be configured to include a portion of the electrical path to the electronic circuitry included in the second module. In more specific embodiments, the upstream drivetrain element abutting against the button cell may be an elongated piston rod which includes an electrically conductive material. In even more specific embodiments, the elongated piston rod may be provided as a composition or aggregate, the composition or aggregate including, in sections, an electrically conductive material and an electrically non-conductive material.

In other embodiments, the expelling mechanism may be entirely allocated to the module including the drug reservoir and the electrical energy source. This, in particular, may be useful to implement a re-usable dose recording concept for mechanical self-contained prefilled pen injection devices. In circumstances, the first module may be a self-contained prefilled mechanical pen injection device that principally provides standalone operation for setting and expelling of a multiple of desired doses from a drug reservoir. Solely, the functionality for electronically monitoring the dose setting and/or expelling manoeuvres in order to trace or monitor the drug administration has to be provided by the second module. In such situation, the electrical energy source may be included with the self-contained prefilled mechanical pen injection device in the same module, preferably within the same external casing structure. From the electrical power source electrical power may be supplied to the second module with the dose recording unit by means of an electromechanical coupling or interface. Simplification may be achieved insofar as the second module will directly power up after attachment to the first module. Additionally, the user may be found relieved from the burden of paying attention to the charge state of the electrical energy source as the lifetime thereof may be chosen to safely extend over the maximum shelf lifetime of the drug in the reservoir. In the same way as with traditional pen injection devices the user may limit, without impacting a fully compliant therapy, on checking for sufficient contents in the drug reservoir and non-expiration of shelf-lifetime. As long as these conditions are met, a user may rely on the dimensioning of the energy source to provide sufficient power for operating the dose recording unit in the second module appropriately.

In another aspect, the present disclosure provides a modularization for an electronically controlled and/or monitored injection device with a first module, the first module comprising a zinc-air cell, particularly being located at one end of the first module, the zinc-air cell having a number of venting openings, whereby a removable seal is provided for covering the venting openings in a first configuration and to reveal the venting openings in a second configuration, a second module, the second module being configured for attachment to the one end of the first module, the second module comprising electronics, whereby
a number of mechanical interfaces is provided on the first module and/or the second module for releasable connecting the first module and the second module together to thereby form a mechanical structure, particularly a rigid mechanical structure,
a seal remover is provided on one of the modules, the seal remover being operable to remove the seal from at least one of the venting openings of the zinc-air cell, and whereby the mechanical interface between the first and the second module is configured to operate the seal remover when the first module is attached to the second module at any of the mechanical interfaces.

It may be advantageous for some purposes that the zin-air cell may be maintained in an inactivated state before the first use of the injection device when the second part is attached to the first part of the drug delivery device. Another advantage may be seen in the comfortable handling by a user since the activation means activate the zinc-air cell when the second part is attached to the first part so that the user does not have to remove the air tight sealing manually as an additional manipulation. If the first part is used as medicament cartridge holder, it can be easily disposed when the cartridge and/or the zinc-air cell is/are empty, and the second part containing the electronics may be reused with another first part.

In an implementation, the first module may be a pen with a distal and a proximal end and a dosage button located at the distal end, and wherein the zinc-air cell is located in the dosage button of the body, particularly on top of the dosage button.

In an alternative implementation, the first module may be a dispense mechanism of the drug delivery device and the zinc-air cell is located in a bearing of the dispense mechanism. The bearing may comprise a cup-like shaped holder for the zinc-air cell and a cup-like shaped cover for imposing and clipping on the holder, wherein the activation means are integrated in the cover.

In yet another alternative implementation, the first module may be a pen with a distal and a proximal end and the zinc-air cell is located in a cell compartment of the first module. The cell compartment may be located at the outside of the first module close to the one end of the first module.

The activation means may comprise at least one pin, which is designed to be used as an electrode for electrically connecting a power supply connector of the electronics with a power supply connector of the zinc-air cell.

In yet another aspect the present disclosure provides an attachment module for a drug delivery device, the attachment module comprising

a flexible body,
a flexible display integrated into the flexible body, and
electronic circuitry being configured to communicate with electronics of the drug delivery device and to control the flexible display depending on a communication between the electronics of the attachment device and the electronics of the drug delivery device.

As an advantage it may be seen that the attachment module allows a versatile use for example by attaching it at different positions to a drug delivery device allowing a better accommodation to user habits such as attaching at a position where a user holding the drug delivery devices does not cover the display of the attachment device. Another advantage is that the attachment device can be easily detached from the drug delivery device and formed so that it may be hold by a user for comfortably reading information shown on the flexible display. Yet another advantage is that the attachment module may be attached to different drug delivery devices due to its flexibility.

The electronic circuitry of the attachment module may be further configured to unlock usage of the drug delivery device depending on the communication.

The electronic circuitry of the attachment module may be configured to control the flexible display such that information derived from the operation of the drug delivery device can be displayed, particularly information on one or more delivered injection dosages, time, holding time, cell status, one or more alarms.

The attachment module may further comprise user input means, wherein the electronic circuitry is configured to process signals generated by the user input means and to control the flexible display and/or the communication with the drug delivery device depending on the processed signals.

The attachment module may further comprise an interface for communication with a computing device, particularly for transmitting data related to the drug delivery device and its usage to the computing device for further processing.

In yet another aspect the present disclosure provides a method for operating a supplementary device for a drug delivery device, wherein the supplementary device comprises electronics having at least one processor and at least one storage and the processor is configured to perform the following steps of the method:

detecting attachment of the supplementary device to the drug delivery device,
detecting a drug delivery with the drug delivery device,
recording the detected drug delivery in the storage, and
generating at least one signal depending on the recording.

An advantage is that a user may be warned by the signal when for example a medicament cartridge of a drug delivery device is empty and should be disposed. The signal may thus remind the user to remove the supplementary device from the drug delivery device if both are coupled together and not to dispose the supplementary device, since it can be reused with a new disposable drug delivery device.

The step of generating at least one signal depending on the recording may comprise one or more of the following:

generating a signal after the last drug delivery with the drug delivery device;
generating a signal before the end position of a drug delivered with the drug delivery device is reached.

The at least one signal may comprise one or more of the following:

an acoustical signal generated with a sound generator of the supplementary device;
a visual signal generated with a visual signal indicator of the supplementary device;
a tactile signal generated with a tactile signal generator of the supplementary device.

### BRIEF DESCRIPTION OF THE FIGURES

The figures show:

FIG. 1: a cutaway illustration of a first embodiment of an injection device;

FIGS. 2A and 2B: cutaway illustrations of a second embodiment of an injection device;

FIG. 2C: a cutaway illustration of an embodiment of a zinc-air cell for application with the embodiments of the injection device;

FIGS. 3A and 3B: cutaway illustrations of a third embodiment of an injection device;

FIGS. 4A and 4B: cutaway illustrations of a fourth embodiment of an injection device;

FIGS. 5A and 5B: cutaway illustrations of a fifth embodiment of an injection device;

FIGS. 6A and 6B: cutaway illustrations of a sixth embodiment of an injection device;

FIGS. 7A and 7B: an illustration of an embodiment of an attachment device for an injection device;

FIG. 8A: a flowchart of an embodiment of a method for operating a supplementary device for a drug delivery device; and

FIG. 8B: an schematic illustration of an embodiment of a supplementary device for an injection device.

### DETAILED DESCRIPTION

In the following, embodiments of the present disclosure will be described with reference to injection devices, particularly a two-part dispensable injection device in the form of a pen. The present disclosure is however not limited to such application and may equally well be deployed with other types of drug delivery devices, particularly with another shape than a pen.

The below described embodiments relate to a modularized pen-shaped injection device comprising two modules, which upon aggregation may build up an electronically controlled and/or monitored injection device, which provides functionality for electronically controlling and/or monitoring expelling of a liquid drug formulation to an external administration site.

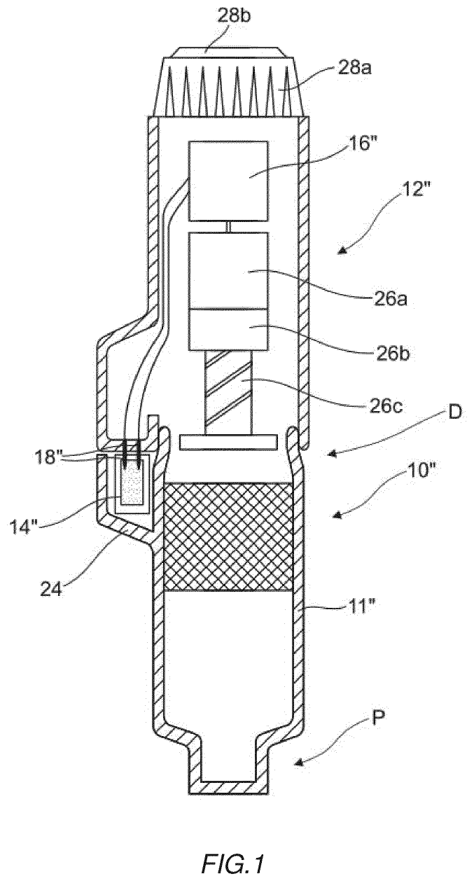

A first embodiment of an injection device will now be described with reference to FIG. 1. The injection device is a two-part device comprising a dispensable injector having a body 10″ in the form of a pen having a proximal end P and a distal end D and a supplementary device 12″ for attachment to the distal end D of the body 10″. The proximal end P of the body 10″ is directed towards the injection site of a patient during an injection while the distal end D is directed away from the injection site.

The body 10″ comprises an outer housing 11″. The outer housing 11″ is an elongate tube. The outer housing 11″ includes a cartridge holder or syringe holder (not shown) which supports a cartridge or syringe containing liquid medicament (not shown) and has a mechanical interface to a supplementary device 12″ containing an electric drivetrain for causing dispensing of the medicament during injection by the body 10″. A cell compartment 24 is located at the outside of the outer housing 11″. The cell compartment 24 is provided for housing a zinc-air cell 14″.

The body 10″ with the zinc-air cell 14″ forms the dispensable part of the injection device, which may be dispensed when the cell and/or cartridge with the liquid medicament is/are empty.

The supplementary device 12″ is designed as an attachment for the distal end D of the body 10″. It comprises an electric drivetrain with a mechanical interface for interfacing with the mechanical interface of the body 10″. The electric drivetrain comprises an electric motor 26a, a gear 26b, and a drive screw 26c. The motor 26a is controlled by electronics 16″ for implementing control and/or measurement functionality of the injection device and which also performs a sensing of the drivetrain in order to ensure the reliability of the drivetrain. The electronics 16″ can also store and/or receive and/or transmit data regarding the usage of the injection device. It may furthermore control a display of the supplementary device 12″. The electronics 16 may for example comprise a microcontroller configured with firmware for measuring and recording usage of the injection device such as time, holding time, cell status, one or more alarms, one or more delivered injection dosages.

Activation means in the form of pins 18″ are located at the attachment side of the supplementary device 12″ opposite to the cell compartment 24 housing the zin-air cell 18″. In this embodiment, the pins 18″ are part of the supplementary device 12″ and integrated in the device 12″.

Usage of the injection device is explained in the following: for the first use, the supplementary device 12″ has to be attached to the body 10″, as shown in FIG. 1. When the supplementary device 12″ is attached to the body 10″ so that the mechanical interfaces are coupled and the drive screw 26c can put a force in a bung in the body 10″ to cause dispense of a medicament, the pins 18″ of the activation means have pierced the air tight sealing of the zinc-air cell 14″ so that air may pass through the pierced air holes in the air tight sealing and activate power supply through the pins 18″ from the now activated zinc-air cell 14″.

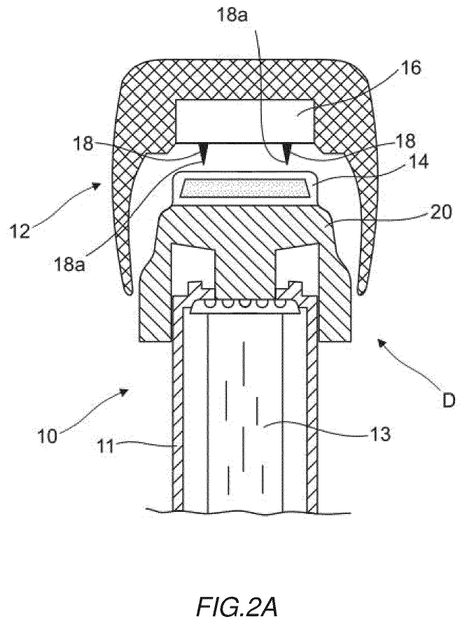

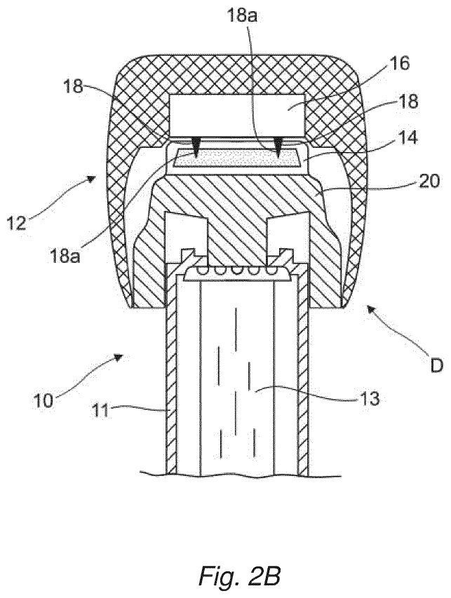

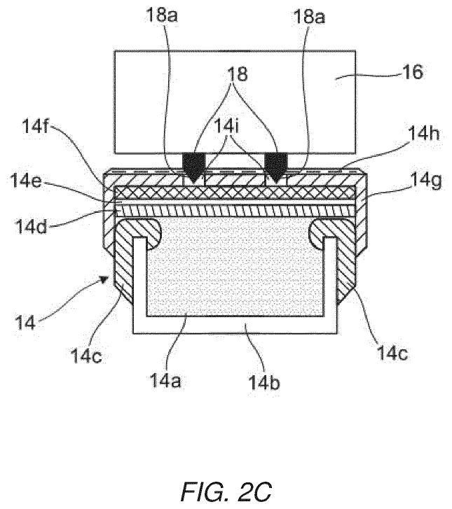

A second embodiment of an injection device will now be described with reference to FIGS. 2A, 2B and 2C. The injection device is a two-part device comprising a dispensable injector having a body 10 in the form of a pen having a proximal end P and a distal end D and a supplementary device 12 for attachment to the distal end D of the body portion 10. The proximal end P of the body 10 is directed towards the injection site of a patient during an injection while the distal end D is directed away from the injection site.

The body 10 comprises an outer housing 11. The outer housing 11 is an elongate tube. The outer housing 11 includes a cartridge holder or syringe holder (not shown) which supports a cartridge or syringe containing liquid medicament (not shown).

The outer housing 11 also houses a dispense mechanism for causing dispensing of the medicament during injection. The dispense mechanism comprises a dosage button 20, which may be pushed onto the outer housing 11. The dosage button 20 is mechanically coupled to a piston 13 of the cartridge (not shown). The dispense mechanism is configured to move the piston axially along the cartridge in a proximal direction to dispense medicament through for example a needle (not shown) at the proximal end. The dosage button 20 may apply a force to the piston 13 in response to an actuation input provided by a user. Here, the actuation input that triggers application of a force to the piston 13 is received by way of the dose dispense or dosage button 20 that is located at the distal end D of the body 10 of the injection device.

A zinc-air (Zn/O₂) cell 14 with an air tight sealing is located on top of the dosage button 20. Due to the air tight sealing, the zinc-air cell is inactive until a first use, when one or more air holes are created in the air tight sealing for activating the zinc-air cell 14.

FIG. 2C shows the zinc-air cell 14 in a cutaway illustration: zinc powder 14a is contained in an anode can 14b and electrically separated from an outer cathode can 14g by an insulator gasket 14c. The outer can 14g is provided with openings 14i. A sealing layer 14h is provided in the outside of the outer can 14g to provide an air tight sealing of the openings 14i. When air holes are created in the sealing layer 14h, air can enter through the openings 14i through a teflon membrane 14f into an air electrode 14e, which is separated from the zinc powder anode 14a by means of a separator 14d. The sealing layer 14h may be for example implemented by air tightly integrating the zinc-air cell 14 in the dosage button 20, for example by injection moulding, wherein the moulding material should be a soft material, for example TPE (thermoplastic elastomer), and/or a thin material to allow piercing for activating the zinc-air cell 14. The piercing allows air to enter through the openings 14i and the Teflon membrane 14f into the air electrode 14e starting the chemical reaction to generate electric power.

The body 10 with the zinc-air cell 14 forms the dispensable part of the injection device, which may be dispensed when the cell and/or cartridge with the liquid medicament is/are empty.

A further part of the injection device is formed by the supplementary device 12, which is designed as a clip-on attachment for the distal end D of the body 10, particularly on the dosage button 20. The supplementary device 12 comprises electronics 16 for implementing control and/or measurement functionality of the injection device. The electronics 16 may for example comprise a microcontroller configured with firmware for measuring and recording usage of the injection device such as time, holding time, cell status, one or more alarms, one or more delivered injection dosages.

Activation means in the form of pins 18 with tips 18a are located at the bottom side of the electronics 16. In this embodiment, the pins 18 are part of the supplementary device 12 and integrated in the device 12.

Usage of the injection device is explained in the following: for the first use, the supplementary device 12 has to be clipped on the body 10, namely on the dosage button 20 as shown in FIGS. 2A and 2B. In FIG. 2A, the supplementary device 12 is not yet completely clipped on the dosage button 20, and FIG. 2B shows it completely clipped on the dosage button 20. As shown in FIG. 2B, when the supplementary device 12 completely covers the dosage button 20, the tips 18a of the pins 18 of the activation means at the bottom side of the electronics 16 have pierced the air tight sealing of the zinc-air cell 14 so that air may pass through the pierced air holes in the air tight sealing and through the openings 14i and the Teflon membrane 14f into the air electrode 14e.

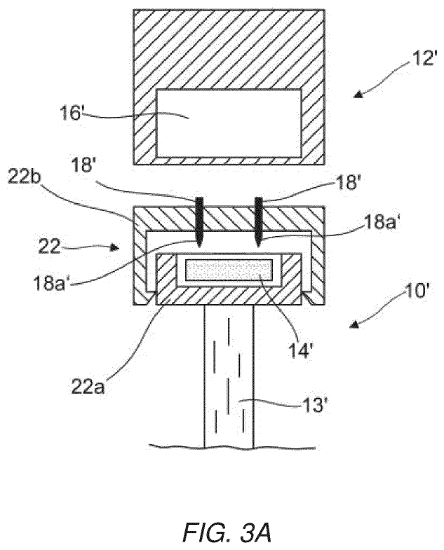

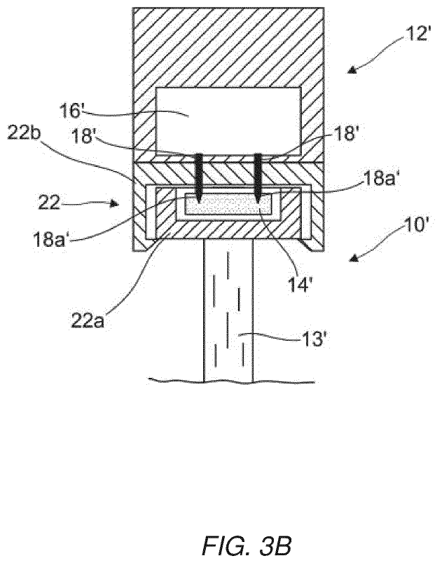

A third embodiment of an injection device will now be described with reference to FIGS. 3A and 3B. The injection device comprises a first part 10′ and a second part 12′ for attachment to one end of the first part 10′.

The first part 10′ is a dispense mechanism of the drug delivery device for causing dispensing of the medicament during injection. The dispense mechanism comprises a bearing 22 for a zinc-air cell 14′ and activation means 18′. The bearing 22 comprises a cup-like shaped holder 22a for the zinc-air cell 14′, which is mechanically coupled to a piston 13′ of the cartridge (not shown). The dispense mechanism is configured to move the piston 13′ axially along a cartridge in a proximal direction to dispense medicament through for example a needle (not shown) at a proximal end. The bearing 22 further comprises a cup-like shaped cover 22b for imposing and clipping on the holder 22a. The bearing 22 forms a button with which a force may be applied to the piston 13′ in response to an actuation input provided by a user. Here, the actuation input that triggers application of a force to the piston 13′ is received by way of the bearing 22 that is located at the distal end D of the body 10′ of the injection device.

The zinc-air (Zn/O₂) cell 14′ with an air tight sealing is located in the holder 22a and covered by the cover 22b. The zinc-air cell 14′ can be for example integrated in the holder 22a by injection moulding. Due to the air tight sealing, the zinc-air cell is inactive until a first use, when one or more air holes are created in the air tight sealing for activating the zinc-air cell 14′. The zinc-air cell 14′ may be similar or even identical to the one shown in FIG. 2C.

The first 10′ with the zinc-air cell 14′ may belong to a dispensable part of the drug delivery device, which may be dispensed when the cell and/or cartridge with the liquid medicament is/are empty.

The second part 12′ is designed as a bung for attachment to the cover 22b and comprises electronics 16′ for implementing control and/or measurement functionality of the injection device. The electronics 16′ may for example comprise a microcontroller configured with firmware for measuring and recording usage of the injection device such as time, holding time, cell status, one or more alarms, one or more delivered injection dosages.

Activation means in the form of pins 18′ with tips 18a′ are integrated in the cover 22b of the first part 10′. Thus, when the cover 22b is clipped on the holder 22a, tips 18a′ of the pins 18′ pierce the air tight sealing of the zinc-air cell 14′, which is then activated. In this embodiment, the pins 18′ are integrated in the first part 10′, namely the cover 22b.

Usage of the injection device is explained in the following: for the first use, the bung 12′ has to be attached to the cover 22b and pressed down to clip the cover 22b on the holder 22b as shown in FIGS. 3A and 3B. In FIG. 3A, the bung 12′ is not yet attached to the cover 22b, and FIG. 3B shows it attached to the cover 22b and pressed down with the cover 22b clipped on the holder 22a. As shown in FIG. 3B, when the bung 12′ is attached to the cover 22b and the cover 22b is clipped on the holder 22a, the tips 18a′ of the pins 18′ of the activation means integrated in the cover 22b have pierced the air tight sealing of the zinc-air cell 14 so that air may pass through the pierced air holes in the air tight sealing and through the openings 14i and the Teflon membrane 14f into the air electrode 14e. The other side of the pins 18′ may pierce the bung 12′ to get into contact with the electronics 16′.

In the above described embodiments, the pins 18, 18′ may also be designed that they could be used as electrodes supplying the electronics 16, 16′.

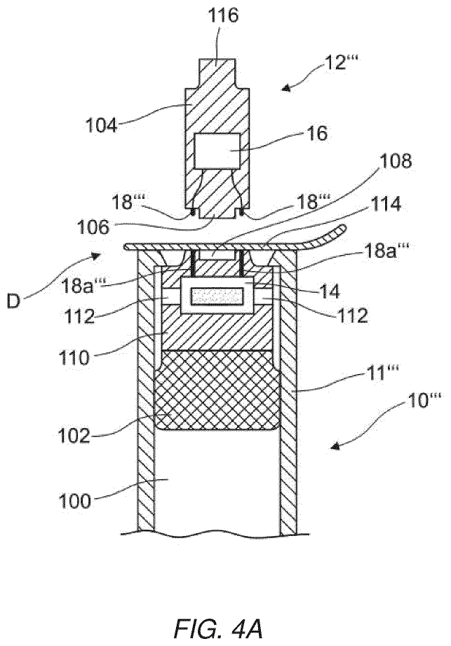

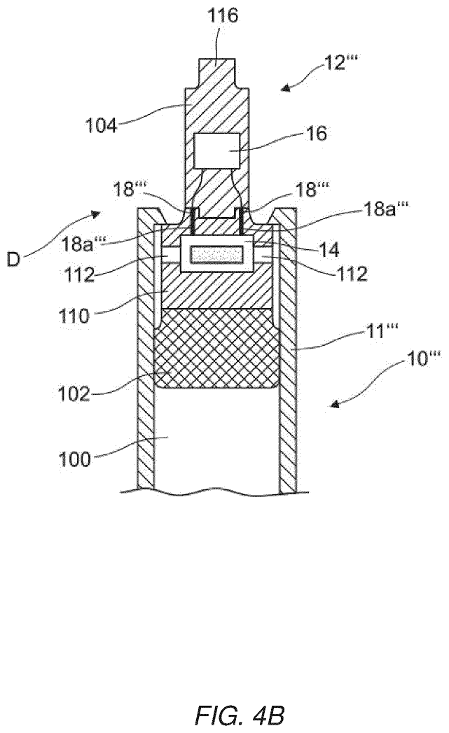

A fourth embodiment of an injection device will now be described with reference to FIGS. 4A and 4B. The injection device is a two-part device comprising a dispensable injector having a body 10′″ in the form of a pen having a proximal end P and a distal end D and a supplementary device 12′″ for attachment to the distal end D of the body portion 10′″. The proximal end P of the body 10 is directed towards the injection site of a patient during an injection while the distal end D is directed away from the injection site.

The body 10′″ comprises a medicament cartridge 11′″ containing a liquid drug formulation, which is included in a section 100 of a glass vial. The section 100 is separated from the exterior space by a rubber bung seal 102, which can be moved along a cylindrical section of the glass vial. Attached to the side of the rubber bung seal 102, which is exposed to the exterior space, is a first part 110 of an electromechanical interface of the body 10′″ and supplementary device 12′″. This first part 110 contains a zinc-air cell 14, two channels 112 extending from the side of the part 110 to the zinc-air cell 14 allowing to pass air to the zinc-air cell 14 from the exterior space, electrical contacts 18a′″ for contacting the zinc-air cell contacts with electrical contacts 18′″ of the supplementary device 12′″, and a recess 108 for mechanically coupling the first part 110 to a second part 104 of the electromechanical interface of the supplementary device 12″. A seal 114 air-tightly covers the open end of the cartridge 11′″ and avoiding activation of the zinc-air cell 14. The outer housing 11′″ is an elongate tube. The first part 110 is hold within the cartridge 11′″ by protrusions provided at the distal end D of the cartridge 11′″ in order to avoid falling out.

The supplementary device 12′″ comprises the second part 104 of the electromechanical interface, which has a coupling side with the electrical contacts 18′″ and a protrusion 106 shaped to fit into the recess 108 of the first part 110. The contacts 18′″ are electrically connected to an electronic circuitry 16 housed in the second part 104 and provided to be powered by the zinc-air cell 14 via the electrical contacts 18′″ and 18a′″.

For using the injection device, the modules 10′″ and 12′″ have to be coupled. This is done by manually removing the seal 114 so that air can pass from the exterior space and the channels 112 to the zinc-air cell 14, and, thus, activating the cell 14. Furthermore, the second part 104 of the electromechanical interface of the supplementary device 12′″ has to be coupled to the first part 110 by attaching the side of the second part 104 comprising the electrical contacts 18′″ and the protrusion 106 to the side of the first part 110 with the recession 108 and the electrical contacts 18a′″, as shown in FIG. 4B.

To expel a portion of the liquid drug formulation, pressure in an axial direction of the first and second parts 110, 104 may be exerted downwards on the section 116 of the second part 104 in order to move downwards the first part 110 and the rubber bung 102.

The electronic circuitry 16 being powered by the activated zinc-air cell 14 via the electrical contacts 18′″,18a′″ may be configured to electronically control and/or monitor the expelling of the liquid drug formulation. For example, the electronic circuitry 16 may be configured to measure the distance of the downwards moving of the second part 104, the first part 110 and the rubber bung 102 and to calculate from the measured distance the expelled amount of the liquid drug formulation. The electronical circuitry 16 may for instance be also configured to control a liquid drug expelling mechanism (not shown) for downwards moving the second part 104, the first part 110 and the rubber bung 102. The mechanism may for comprise an electric motor and a gear with a drive screw for driving the second part 104 downwards (similar for example to the mechanism 26a, 26b, 26c of the embodiment shown in FIG. 1).

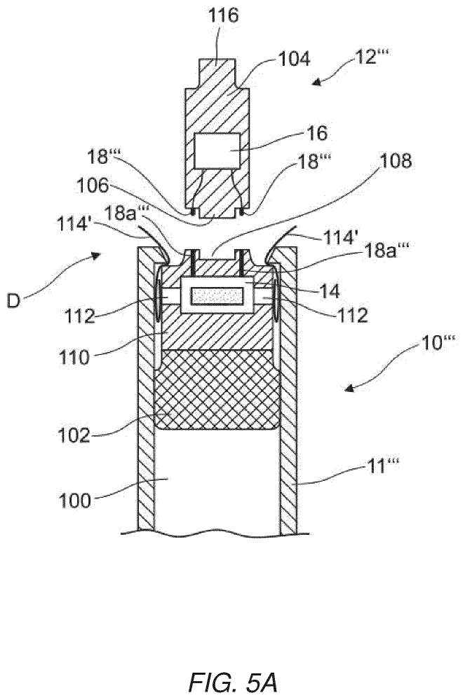

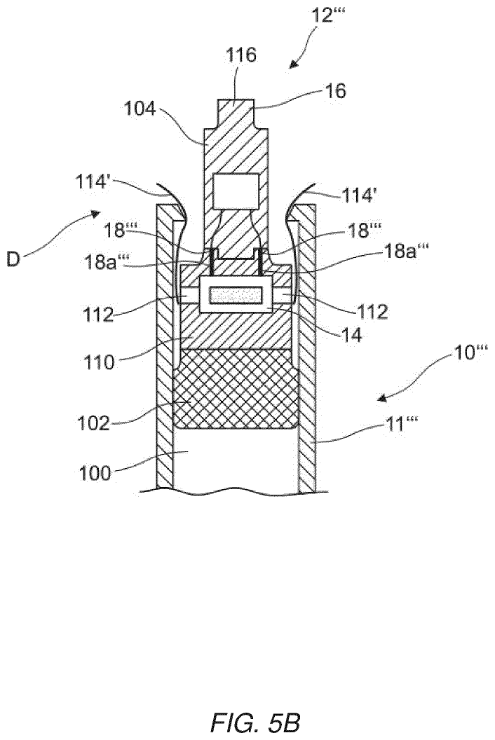

A fifth embodiment of an injection device will now be described with reference to FIGS. 5A and 5B. The fifth embodiment is similar to the fourth embodiment and differs merely in the implementation of the air tightly sealing of the zinc-air cell 14: the sealing comprises two seals 114′ each covering one of the entrances of the two channels 112 extending from the side of the part 110 to the zinc-air cell 14 allowing to pass air to the zinc-air cell 14 from the exterior space. The seals 114′ may extend beyond the edge of the medicament cartridge 11′″ and may be fixed to the edge, for example adhered to the edge. The other ends of the seals 114′ may be removably adhered over the entrances of the two channels 112 so that when the first part 110 is moved downwards, the seals 114′ are pulled away from the entrances of the two channels 112, thus, removing the air tight sealing of the channels 112. Thus, upon first usage of the injection device, i.e. when both modules 10′″ and 12′″ are coupled together and the second part 104, the first part 110, and the rubber bung 102 are moved downwards to expel a dosage of the liquid drug formulation, the seals 114′ are pulled from the entrances of the two channels 112 so that air can pass from the exterior space and the channels 112 to the zinc-air cell 14 and, thus, activate the cell 14.

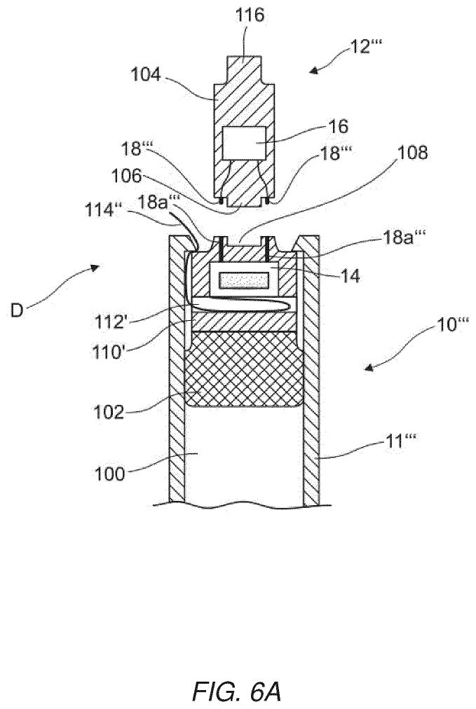

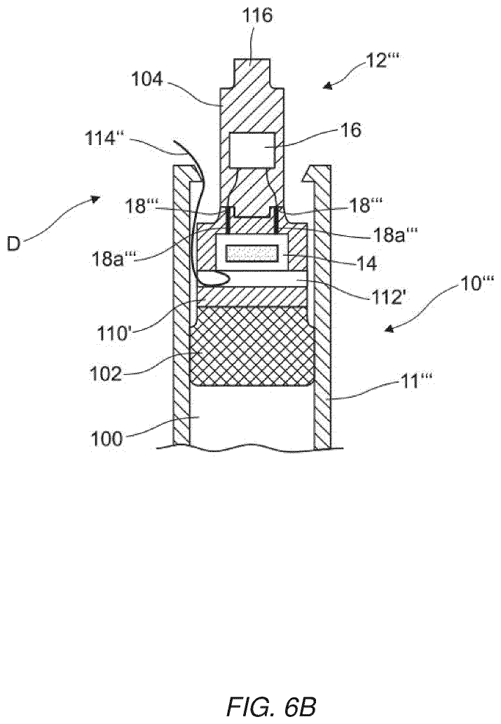

FIGS. 6A and 6B show a sixth embodiment of an injection device, which will now be described with reference to these Figures. Also, the sixth is similar to the fourth and fifth embodiments, but it differs in the implementation of the first part 110′ and the air tightly sealing of the zinc-air cell 14 located within the first part 110′. As can be seen in the FIGS. 6A and 6B, the first part 110′ comprises one channel 112′ extending from the left to the right side of the part 110′. The zinc-air cell 14 is located in the part 110′, wherein one side of it is exposed to the channel 112′. Thus, when air enters the channel 122′ it may pass through the channel to the exposed side of the zinc-air cell 14 and may activate the cell 14. The exposed side of the cell 14 is in an unused condition covered by a seal 114″, for example removably adhered to the exposed side and extending through the channel 112′ beyond the edge of the medicament cartridge 11′″ and may be fixed to the edge, for example adhered to the edge. When the first part 110′ is moved downwards, the seal 114′ is pulled away from the exposed side of the zinc-air cell 14 so that air passing through the channel 112′ can activate the zinc-air cell 14. Thus, upon first usage of the injection device, i.e. when both modules 10′″ and 12′″ are coupled together and the second part 104, the first part 110′, and the rubber bung 102 are moved downwards to expel a dosage of the liquid drug formulation, the seal 114″ is pulled from the exposed side of the zinc-air cell 14 so that air can pass from the exterior space and the channel 112′ to the zinc-air cell 14 and, thus, activate the cell 14.

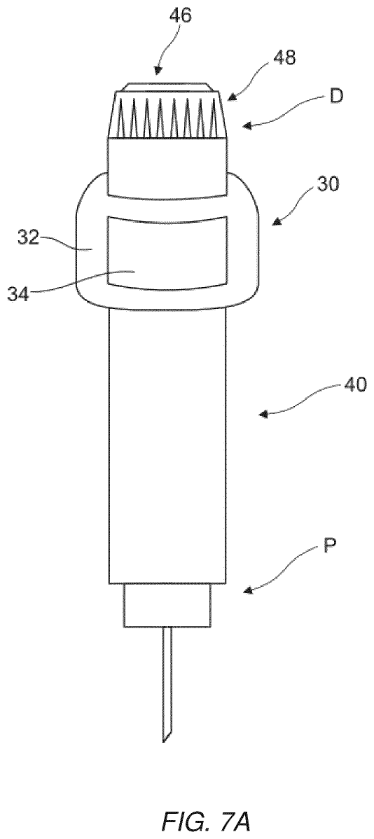

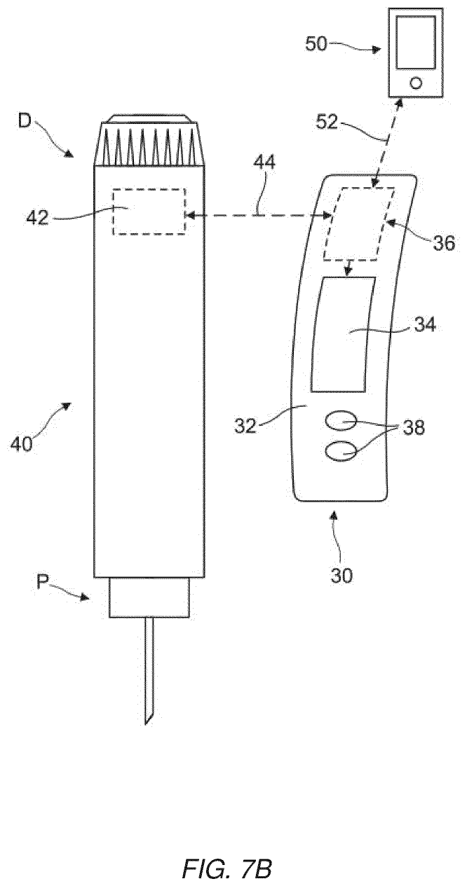

In the following, an embodiment of an attachment device 30 for a drug delivery device 40 is described with reference to FIGS. 7A and 7B.

The drug delivery device 40 may be a pen shaped injection device having an elongate body 40 with a distal end D and a proximal end P. At the proximal end P, a syringe may be provided for injection of a medicament from a cartridge inside the body 40 into a patient's body. At the distal end D, a dispense button 46 and a dosage selector 48 may be provided. The dosage to be injected may be selected with the dosage selector 48, and the dispense may be initiated by a patient by pressing the dispense button 46. An internal mechanism may then be triggered by the pressing of the dispense button 46 to inject the selected dosage through the syringe into the patient's body.

The drug delivery device 40 may further house electronics 42 adapted to perform tasks such as for example measuring a delivered injection dosage, time, holding time, cell status, and/or outputting one or more alarms, for example when the medicament cartridge and/or the cell is empty. The electronics 42 may be further adapted for communication and data exchange with another electronics, particularly via radio transmission such as for example according to the Bluetooth® standard or a near field communication (NFC) standard.

The attachment device 30 for the drug delivery device 40 comprises a flexible body 32, in which a flexible display 34, particularly a flexible OLED (Organic Light Emitting Diode) display, is integrated. The attachment device 30 is bendable so that it can be twisted around the body 40 of the drug delivery device and placed at a position preferred by a user. For example, a user can position the attachment device 30 at the body 40 so that the display 34 is not covered by her/his hands when using the drug delivery device.

FIG. 7B shows the attachment device 30 detached from the body 40 of the drug delivery device and bended in a flat handheld mode, in which it can be for example hold by a user or laid down on a table.

As shown in FIG. 7B, the attachment device 30 can also comprise user input means 38 in form of buttons and/or the flexible display 34 may comprise touch screen functionality in order to receive user inputs. Electronics 36 is integrated in the flexible body 32, which is configured to control the display 34, to process signals generated by the user inputs means 38, and/or to communicate with the electronics 42 of the drug delivery device 40 and/or a computing device 50 such as a mobile computing device, for example a smartphone, a laptop and/or a tablet computer. The communication 44 with the electronics 42 of the drug delivery device may be wired or wireless. For a wired communication, the electrical contacts may by integrated in the flexible body and also electrical contacts may be integrated in the body 40 of the drug delivery device, for example several contacts at different positions at the body 40 in order to allow to place the attachment device 30 at different positions at the body 40. Electrical contacts may be also provided on the flexible body 32 for connecting the attachment device 30 with the computing device 50, for example a micro-USB (Universal Serial Bus) connector. A wireless communication 44 with the electronics 42 may be performed via a particularly short-range radio communication such as according to the Bluetooth® standard or a near field communication (NFC) standard. A wireless communication 52 with the computing device 50 may be performed via a short-range radio communication such as according to the Bluetooth® standard or a near field communication (NFC) standard, or via a wider range radio communication such as according to the IEEE802.11 standard.

A communication 44 between the electronics 36 of the attachment device 30 and the electronics 42 of the drug delivery device 40 can established upon a user input via the user input means 38 and/or via a touch screen input if the display 34 has touch screen functionality and/or automatically when the attachment device 30 is twisted around the body 40. For example, a switch integrated in the flexible body 32 may be activated upon bending the body 32 so that the electronics 36 is switched into a mode in which a communication 44 with the electronics 42 of the drug delivery device 40 can be established.

The electronics 36 of the attachment device 30 may receive data from and transmit data to the electronics 42 of the drug delivery device 40 via an established communication 44. For example, the electronics 36 may receive data regarding the use of the drug delivery device 40, such as delivered injection dosages, time, holding time, device's 40 cell status, one or more alarms, and/or transmit data to control use of the drug delivery device 40, such as an unlocking code allowing a user to operate the drug delivery device 40.

The electronics 36 is configured to control the display 34 such that information received via a communication 44 from the drug delivery device 40 is displayed. The electronics 36 may be further configured to process signals generated by the user inputs means 38 and/or a touch screen. Also, the electronics 36 may be configured to control the communication 44 depending on the processed signals generated by the user inputs means 38 and/or a touch screen. For example, after attaching the device 30 to the drug delivery device 40, a user may press one of the user input means 38 in order to establish a communication 44 and after establishment of the communication 44 send an unlock code via the communication 44 to the electronics 42 of the drug delivery device 40 to enable an injection by a patient. The electronics 36 may be further configured to enable customization of the information displayed on the display 34, for example the arrangement and/or kind of displayed information.

Furthermore, the attachment device 30 can be configured to be assigned to a certain user by means of a user identification stored by the electronics 36 so that it can only be used by that user and not by other users. In order to identify the user, the electronics 36 may be configured to request a code a user has to input via the user input means 38 or a touch screen before allowing to use the attachment device 30.

The electronics 36 of the attachment device 30 may be further configured to store data received from the electronics 42 of the drug delivery device 40. The stored data can then be transmitted via a communication 52 to a computing device 50 for further processing, for example evaluation and/or storing in a therapy ID card of a patient. The communication 52 can be also configured to customize the attachment device 30, for example to adapt it to user requirements such as assigning it to a certain user, customizing the information to be displayed on the display 34. For a comfortable customization, the computing device 50 may be configured by a dedicated software such as an app for programming the attachment device 30 and/or evaluating data received from the electronics 36.

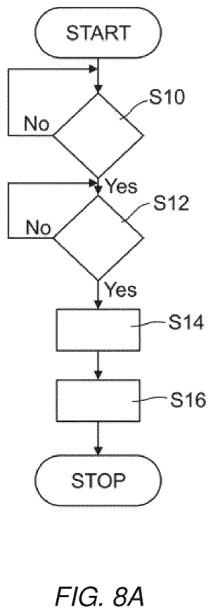

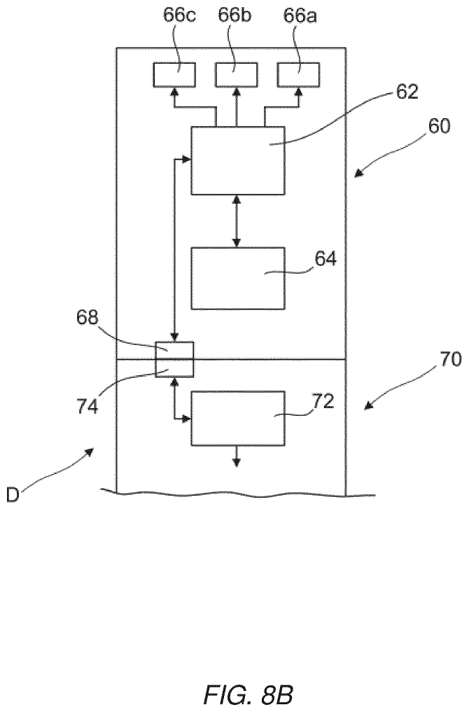

Now, an embodiment of a method for operating a supplementary device 60 for a drug delivery device 70 is described with reference to FIGS. 8A and 8B. The supplementary device 60 is a re-usable add-on device for a disposable drug delivery device 70. Typically, the supplementary device 60 may contain some electronics for controlling and monitoring the usage of the drug delivery device 60. For example, it may contain control electronics to assist a user of the drug delivery device 70 in the correct usage, particularly the injection of a medicament. It may also comprise monitoring electronics for determining the number of usage of the drug delivery device 70, for example for recording the injected dosages of a medicament, the time and date of each injection.

The method serves to output at least one signal depending on a recording of drug deliveries with the drug delivery device 70. The outputting of a signal may assist a user of the disposable drug delivery device 70 in preventing a disposal of the reusable supplementary device 60, or in other words warn a user to detach the supplementary device 60 from the drug delivery device 70 when disposing the later.

The method is implemented as an algorithm to be executed by the supplementary device 60. The algorithm may signal a user of the disposable drug delivery device 70 recordings of detected drug deliveries, particularly signal when the disposable drug delivery device 70 is empty.

An embodiment of the algorithm is explained in detail with reference to the flowchart shown in FIG. 8A and the schematic illustration of the supplementary device 60 attached to the distal end D of the drug delivery device 70 shown in FIG. 8B.

The algorithm is implemented as firmware stored in a memory 64 of the supplementary device 60, which is executed by a processor 62. The supplementary device 60 may comprise several signalling means, particularly a sound generator 66a such as a loudspeaker, a visual signal indicator 66b such as a LED (Light Emitting Diode), a tactile signal generator 66c such as a vibrating alert motor.

A sensor (not shown) may be provided to detect an attachment of the supplementary device 60 to the drug delivery device 70. The sensor may be also implemented as a contact 68, which can be contacted with a counter-contact 74 of the drug delivery device 70, or as a contactless sensor, for example a hall sensor, which may detect a magnetic field created by a magnet of the drug delivery device 70.

The drug delivery device 70 may comprise electronics 72 for measuring drug deliveries, for example an electronic counter for simply counting the delivered dosages, and the counter-contact 74 for the contact 68. Data exchange between the processor 62 and the electronics 72 may be wireless or wired as shown in FIG. 8B by means of the contacts 68, 74 through which a wired data link may be established between the processor 62 and the electronics 72 upon attachment of the supplementary device 60 to the drug delivery device 70.

In a first sept S10 after starting, the algorithm checks if the supplementary device 60 is attached to the drug delivery device 70 by means of the sensor. For example, the processor 62 receive a signal from the electronics 72 via a wired data link established via the contacts 68 and 74. If a wireless sensor is used, the processor 62 may receive a signal from the sensor signalling an attachment of the supplementary device 60 to the drug delivery device 70, for example a signal from a hall sensor detecting a magnetic field in its vicinity. If attachment of the supplementary device 60 to the drug delivery device 70 is detected in step S10, the algorithm continues with the second step S12.

In step S12, the algorithm detects if a drug is/was delivered with the drug delivery device 70. Particularly, the processor 62 can request information on drug delivery from the electronics 72 of the drug delivery device 70, for example via a data link established via the contacts 68 and 74. It is also possible that the processor 62 detects a drug delivery by means of a sensor (not shown) integrated in the supplementary device 60 or the drug delivery device 70 and provided for detecting a drug delivery. Such a sensor could be used when only the supplementary device 60 contains electronics, and the disposable drug delivery device 70 contains only a mechanics for dispensing a medicament from a cartridge. The detected drug delivery may be a count of every dispensed dosage, for example x dispensed dosages, a count for total dispensed dosage, for example y total dispensed dosage, or a last dosage counter, for example xth dispensed dosage. It is also possible that a counter starts with the maximum number of dosages contained in a cartridge and is counted down with every dispensed dosage, and the detected drug delivery is the actually read out counter value.

The detected drug delivery is in a third step S14 recorded in the storage 64 by the processor 62. The recording may also comprise a date and/or time stamp, and a unique identifier of the drug delivery device 70 or the medicament cartridge contained in the device 70 in order to be able to assign the recordings to the drug device 70. With the unique identifier the supplementary device could be used with different drug delivery devices 70.

After the recording step S14, the algorithm continues in a fourth step S16 with generating at least one signal depending on the recording performed in the previous step S14. In detail, the processor may check in step S16 whether the recording performed in step S14 fulfils one or more predetermined criteria.

One criterion may be that the last dosage was delivered by the drug delivery device 70. Another criterion may comprise the position before the end position is reached in the drug delivery device, for example when a medicament cartridge contains only remaining dosage.

The processor may check the fulfilment of the criteria for example by comparing the detected drug delivery recorded in step S14 with a maximum number of possible dosages, or if a count-down counter falls below a predefined threshold such as 1 or 2 (for number of remaining dosages), or if a dosage counter exceeds a predefined threshold such as a maximum number of dosages.

If the processor 62 detects that the one or more predetermined criteria are fulfilled, it may generate a control signal for one or more of the signalling means. For example, the processor 62 may control the sound generator 66a to generate a sound signal, the visual signal indicator 66b to generate a visible signal, and/or the tactile signal generator 66c to generate a tactile feedback signal such as a vibration of the supplementary device 60. Thus, a user may note that the last dosage contained in the cartridge of the drug delivery device 70 was dispensed, or only one further dosage remains for injection, and the drug delivery device 70 has to be replaced now or soon with a new one and the supplementary device 60 containing the electronics for controlling the drug dispense with the drug delivery device 70 should be removed from the drug delivery device 70 and not be disposed.

1-26. (canceled)

27. A drug delivery device with a first and/or a second module, wherein the first and/or second modules are configured for, when aggregated, building up an electronically controlled and/or electronically monitored pen-shaped injection device and to thereby provide functionality for electronically controlled and/or monitored expelling of a liquid drug formulation to an external administration site, the drug delivery device comprising:
the first module comprising a liquid drug reservoir, a first, downstream, portion of a liquid drug expelling mechanism configured to expel a portion of liquid drug from the liquid drug reservoir when actuated, and a self-contained electrical energy source; and/or
the second module comprising a second, upstream, portion of the liquid drug expelling mechanism and an electronic circuitry, the electronic circuitry being configured for controlling and/or monitoring of a state and/or operation of any of the first or second portion of the liquid drug expelling mechanism,
wherein electromechanical interfaces are provided on the first and/or second modules, the electromechanical interfaces being configured to:
releasably connect the first module and the second module to thereby form a connected mechanical structure;
operationally connect the first, downstream, portion of the liquid drug expelling mechanism of the first module to the second, upstream, portion of the liquid drug expelling mechanism of the second module; and
provide an electrical path for supplying energy from the self-contained electrical energy source included in the first module to the electronic circuitry included in the second module.

28. The drug delivery device of claim 27, wherein the first and/or second module are/is configured for aggregating into an axial layout, wherein the axial layout comprises the liquid drug filled reservoir, the liquid drug expelling mechanism, and the self-contained electrical energy source arranged along a longitudinal axis defined by a pen-shaped external appearance of the aggregated first and second module.

29. The drug delivery device of claim 27, wherein the first module comprises a standard medicament cartridge with the liquid drug formulation included in a section of a glass vial, the section of the glass vial being separated from an exterior space by a rubber bung seal, the rubber bung seal being movable along a cylindrical section of the glass vial as an ultimate end of the first, downstream, portion of the liquid drug expelling mechanism in the first module for transforming mechanical drive force in the liquid drug expelling mechanism into liquid pressure in the separated glass vial section.

30. The drug delivery device of claim 29, wherein the self-contained electrical energy source is a button cell located adjacent to a surface of the rubber bung seal, wherein the surface of the rubber bung seal faces the exterior space.

31. The drug delivery device of claim 30, wherein the button cell comprises a ridged external structure suitable for transferring an axial load from an abutting drivetrain member into the rubber bung seal.

32. The drug delivery device of claim 30, wherein the button cell is a flat-cylindrical button cell arranged to contact an externally-facing end of the rubber bung seal with one flat circular pole and to provide an opposite circular pole face as an incoming load plate to an adjacent upstream drivetrain element of an overall expelling drivetrain.

33. The drug delivery device of claim 30, wherein the button cell is a flat-cylindrical button cell, and an external edge surrounding an opposite circular pole of the button cell is used as an input load support for an upstream drivetrain element in order to avoid transfer of mechanical load over a seal of the button cell or through its internal structure.

34. The drug delivery device of claim 32, wherein the upstream drivetrain element abutting against the button cell is configured to include a portion of the electrical path to the electronic circuitry included in the second module.

35. The drug delivery device of claim 32, wherein the upstream drivetrain element abutting against the button cell is an elongated piston rod which comprises an electrically conductive material.

36. The drug delivery device of claim 35, wherein the elongated piston rod is provided as a composition or aggregate, the composition or aggregate comprising, in sections, an electrically conductive material and an electrically non-conductive material.

37. An electronically controlled and/or monitored injection device with
a first module, the first module comprising a zinc-air cell, the zinc-air cell having a plurality of venting openings, wherein a removable seal is provided for covering the plurality of venting openings in a first configuration and to reveal the plurality of venting openings in a second configuration; and
a second module, the second module being configured for attachment to one end of the first module, the second module comprising electronics, wherein a plurality of mechanical interfaces are provided on the first module and/or the second module for releasably connecting the first module and the second module together to form a mechanical structure,
wherein a seal remover is provided on one of the first and second modules, the seal remover being operable to remove the removable seal from at least one of the plurality of venting openings of the zinc-air cell, and wherein one of the plurality of mechanical interfaces between the first and the second module is configured to operate the seal remover when the first module is attached to the second module at the mechanical interface.

38. The injection device of claim 37, wherein the zinc-air cell is located at one end of the first module.

39. The injection device of claim 37, wherein the first module is pen-shaped with a distal end, a proximal end, and a dosage button located at the distal end, and wherein the zinc-air cell is located on top of the dosage button.

40. The injection device of claim 37, wherein the first module is a dispense mechanism of the injection device and the zinc-air cell is located in a bearing of the dispense mechanism.

41. The injection device of claim 40, wherein the bearing comprises a cup-like shaped holder for the zinc-air cell and a cup-like shaped cover for imposing and clipping on the cup-like shaped holder, wherein an activation mechanism are integrated in the cup-like shaped cover.

42. The injection device of claim 37, wherein the first module is pen-shaped with a distal end and a proximal end, and the zinc-air cell is located in a cell compartment of the first module.

43. The injection device of claim 42, wherein the cell compartment is located outside of the first module and close to the one end of the first module.

44. The injection device of claim 37, wherein an activation mechanism comprise at least one pin, which is designed to be used as an electrode for electrically connecting a power supply connector of the electronics with a power supply connector of the zinc-air cell.

45. An attachment module for a drug delivery device with a first and/or a second module, wherein the first and/or second modules are configured for, when aggregated, building up an electronically controlled and/or electronically monitored pen-shaped injection device and to thereby provide functionality for electronically controlled and/or monitored expelling of a liquid drug formulation to an external administration site, the drug delivery device comprising:
the first module comprising a liquid drug reservoir, a first, downstream, portion of a liquid drug expelling mechanism configured to expel a portion of liquid drug from the liquid drug reservoir when actuated, and a self-contained electrical energy source; and/or
the second module comprising a second, upstream, portion of the liquid drug expelling mechanism and an electronic circuitry, the electronic circuitry being configured for controlling and/or monitoring of a state and/or operation of any of the first or second portion of the liquid drug expelling mechanism,
wherein electromechanical interfaces are provided on the first and/or second modules, the electromechanical interfaces being configured to:
releasably connect the first module and the second module to thereby form a connected mechanical structure;
operationally connect the first, downstream, portion of the liquid drug expelling mechanism of the first module to the second, upstream, portion of the liquid drug expelling mechanism of the second module; and
provide an electrical path for supplying energy from the self-contained electrical energy source included in the first module to the electronic circuitry included in the second module,
the attachment module comprising:
a flexible body;
a display integrated into the flexible body; and
electronic circuitry being configured to communicate with electronics of the drug delivery device and to control the display depending on a communication between the electronics of the attachment module and the electronics of the drug delivery device.

46. The attachment module of claim 45, wherein the electronic circuitry is further configured to unlock usage of the drug delivery device depending on the communication.

47. The attachment module of claim 45, wherein the electronic circuitry is configured to control the display such that information derived from an operation of the drug delivery device can be displayed, the information comprises at least one or more delivered injection dosages, time, holding time, cell status, or one or more alarms.

48. The attachment module of claim 45, further comprising user input mechanism, wherein the electronic circuitry is configured to process signals generated by the user input mechanism and to control a display and/or the communication with the electronics of the drug delivery device depending on the processed signals.

49. The attachment module of claim 45, further comprising an interface for communication with a computing device and for transmitting data related to the drug delivery device and its usage to the computing device for further processing.

50. A method for operating a supplementary device for a drug delivery device with a first and/or a second module, wherein the first and/or second modules are configured for, when aggregated, building up an electronically controlled and/or electronically monitored pen-shaped injection device and to thereby provide functionality for electronically controlled and/or monitored expelling of a liquid drug formulation to an external administration site, the drug delivery device comprising:
the first module comprising a liquid drug reservoir, a first, downstream, portion of a liquid drug expelling mechanism configured to expel a portion of liquid drug from the liquid drug reservoir when actuated, and a self-contained electrical energy source; and/or
the second module comprising a second, upstream, portion of the liquid drug expelling mechanism and an electronic circuitry, the electronic circuitry being configured for controlling and/or monitoring of a state and/or operation of any of the first or second portion of the liquid drug expelling mechanism,
wherein electromechanical interfaces are provided on the first and/or second modules, the electromechanical interfaces being configured to:
releasably connect the first module and the second module to thereby form a connected mechanical structure;
operationally connect the first, downstream, portion of the liquid drug expelling mechanism of the first module to the second, upstream, portion of the liquid drug expelling mechanism of the second module; and
provide an electrical path for supplying energy from the self-contained electrical energy source included in the first module to the electronic circuitry included in the second module,
wherein the supplementary device comprises electronics having at least one processor and at least one storage and the processor is configured to perform the following steps of the method:
detecting attachment of the supplementary device to the drug delivery device;
detecting a drug delivery with the drug delivery device;
recording the detected drug delivery in the at least one storage; and
generating at least one signal depending on the recording.

51. The method of claim 50, wherein the step of generating at least one signal depending on the recording comprises one or more of the following:
generating a signal after the last drug delivery with the drug delivery device; or
generating a signal before an end position of a drug delivered with the drug delivery device is reached.

52. The method of claim 50, wherein the at least one signal comprises one or more of the following:
an acoustical signal generated with a sound generator of the supplementary device;
a visual signal generated with a visual signal indicator of the supplementary device; or
a tactile signal generated with a tactile signal generator of the supplementary device.

53. The drug delivery device of claim 27, wherein the self-contained electrical energy source comprises an electrochemical cell or battery.

54. The drug delivery device of claim 27, wherein the connected mechanical structure is a rigidly connected mechanical structure.
